(19) United States
(12) Patent Application Publication
(10) Pub. No.: US 2007/0078484 A1
(43) Pub. Date: Apr. 5, 2007

(54) Gentle touch surgical instrument and method of using same

(76) Inventors: Joseph Talarico, Chicago, IL (US); Dan M. Mihai, Hanover Park, IL (US); Douglas A. Rathburn, Dubuque, IA (US)

Correspondence Address: WALLENSTEIN & WAGNER, LTD., 311 SOUTH WACKER DRIVE, 53RD FLOOR, CHICAGO, IL, 60606, US

(21) Appl. No.: 11/242,304

(22) Filed: Oct. 3, 2005

(51) Int. Cl. A61B 17/00

(52) U.S. Cl. 606/205

(57) ABSTRACT

A surgical grasper is provided. The grasper comprises a handle, two jaws operably connected to the handle, which jaws can be actuated by the handle, and a sensor. A surgical grasper for use in robotic surgery is also provided. The grasper comprises a shaft, two jaws at a distal end of the shaft, which jaws can be actuated in response to a robot command, and a sensor. A method for measuring an amount of force being applied by a jaw of a grasper is also provided. The method comprises the steps of: providing a grasper comprising a handle and two jaws operably connected to the handle, which jaws can be actuated by the handle; providing a sensor on the grasper; and, providing for measuring an amount of force being applied to the sensor. A method for measuring an amount of force being applied by a jaw of a grasper for use in robotic surgery is also provided. The method comprises the steps of: providing a grasper for use in robotic surgery, the grasper comprising a shaft and two jaws at a distal end of the shaft, which jaws can be actuated responsive to a robot command; providing a sensor; and, providing for measuring an amount of force being applied to the sensor. A surgical feedback system is also provided. The surgical feedback system comprises a surgical grasper capable of taking a force measurement and a data concentrator coupled to the grasper via a wired or wireless interface using a first data transmission protocol with internal storage. A method for obtaining surgical feedback is also provided. The method comprises the steps of: providing a surgical grasper capable of taking a force measurement; and, providing a data concentrator coupled to the grasper via a wired or wireless interface using a first data transmission protocol with internal storage.

### CROSS-REFERENCE TO RELATED APPLICATIONS

Not Applicable.

### FEDERALLY SPONSORED RESEARCH OR DEVELOPMENT

Not Applicable.

### TECHNICAL FIELD

The present invention relates generally to a surgical instrument and method of using same, and more specifically to a force- or pressure-sensitive surgical instrument and a method of measuring a force or pressure being applied by a surgeon with the force- or pressure-sensitive surgical instrument, and the transmission of force or pressure data in real-time to a concentrator device for storage, playback, and further transmission to a visual display, a patient monitoring station or system, or a Healthcare Information System.

### BACKGROUND OF THE INVENTION

Various types of surgical instruments and methods of using same are well known in the art. While such surgical instruments and methods of using same according to the prior art provide a number of advantageous features, they nevertheless have certain limitations. The present invention seeks to overcome certain of these limitations and other drawbacks of the prior art, and to provide new features not heretofore available. A full discussion of the features and advantages of the present invention is deferred to the following detailed description, which proceeds with reference to the accompanying drawings.

### SUMMARY OF THE INVENTION

The present invention generally provides a surgical grasper comprising a handle and two jaws operably connected to the handle. The jaws can be actuated by the handle. A sensor is located on an inner surface of one or both of the jaws for direct measurement of an amount of pressure or force being applied with the grasper. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the piezoelectric sensor or piezoelectric crystal is used, then a resistor having a fixed resistance is connected in series with the piezoelectric sensor located on an inner surface of one or both jaws or remotely inside the handle. A voltage drop is measurable across the fixed resistor, which voltage drop corresponds to an amount of change in force being applied to the piezoelectric sensor. A voltage integration circuit converts the force change signal generated by the piezoelectric sensor into a signal proportional to the absolute value of the force being applied. This voltage integration circuit is not necessary if the sensor technology is based on a true pressure- or force-reading principle. An audio alert and/or a visual signal corresponding to an amount of force or pressure being applied to the sensor can be included.

According to another embodiment, a surgical grasper comprises a handle and two jaws operably connected to the handle. The jaws can be actuated by the handle. A sensor is located on or inside the handle for indirect measurement of an amount of pressure or force being applied with the grasper at an actuator level. If this indirect measurement approach is used, a calibration procedure is implemented at manufacturing time to determine and store a calibration profile inside a non-volatile memory located in the instrument's handle which will be used to convert, in real-time, the indirect measurements taken into the force or pressure values applied at the jaws. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the piezoelectric sensor or piezoelectric crystal is used, then a resistor having a fixed resistance is connected in series with the piezoelectric sensor located remotely inside the handle. A voltage drop is measurable across the fixed resistor, which voltage drop corresponds to an amount of change in force being applied to the piezoelectric sensor. A voltage integration circuit converts the force change signal generated by the piezoelectric sensor into a signal proportional to the absolute value of the force being applied. This voltage integration circuit is not necessary if the sensor technology is based on a true pressure- or force-reading principle. An audio alert and/or a visual signal corresponding to an amount of force or pressure being applied to the sensor can be included.

According to still another embodiment, a surgical grasper is specifically designed for use in robotic surgery. The grasper comprises a shaft with two jaws at a distal end of the shaft. The jaws can be actuated in response to a robot command. A sensor is located on an inner surface of one or both of the jaws for direct measurement of an amount of pressure or force being applied with the grasper. The sensor can be any type of force or pressure sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the sensor is a piezoelectric sensor or piezoelectric crystal, a resistor having a fixed resistance is connected in series with the piezoelectric sensor, wherein a voltage drop is measurable across the fixed resistor, which voltage drop corresponds to an amount of change in force being applied to the piezoelectric sensor. A voltage integration circuit converts the force change signal generated by the piezoelectric sensor into a signal proportional to the absolute value of the force being applied. In this embodiment, the measured voltage drop or the processed voltage can be fed back to the robot for use in adjusting the amount of force being applied by the jaws. A visual or audio signal corresponding to an amount of force or pressure being applied to the sensor can be included.

According to yet another embodiment, a surgical grasper is specifically designed for use in robotic surgery. The grasper comprises a shaft with two jaws at a distal end of the shaft. The jaws can be actuated in response to a robot command. A sensor is located at a proximal end of the shaft, at an actuator, or on a wrist of a robot arm for indirect measurement of an amount of pressure or force being applied with the grasper at the actuator level. If the indirect measurement approach is used, a calibration procedure is implemented at manufacturing time to determine and store a calibration profile inside a non-volatile memory located remotely from the grasper's distant end of the shaft which will be used to convert, in real-time, the indirect measurements taken into the force or pressure values applied at the jaws. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the piezoelectric sensor or piezoelectric crystal is used, then a resistor having a fixed resistance is connected in series with the piezoelectric sensor located remotely inside the handle. A voltage drop is measurable across the fixed resistor, which voltage drop corresponds to an amount of change in force being applied to the piezoelectric sensor. A voltage integration circuit converts the force change signal generated by the piezoelectric sensor into a signal proportional to the absolute value of the force being applied. This voltage integration circuit is not necessary if the sensor technology is based on a true pressure- or force-reading principle. In this embodiment, the measured voltage drop or the processed voltage can be fed back to the robot for use in adjusting the amount of force being applied by the jaws. A visual or audio signal corresponding to an amount of force or pressure being applied to the sensor can be included.

According to still another embodiment, a method for measuring an amount of force being applied by the jaws of a grasper is provided. The method comprises the step of providing a grasper comprising a handle and two jaws operably connected to the handle, which jaws can be actuated by the handle. The method further comprises the steps of providing a sensor on an inner surface of one or both of the jaws of the grasper, and providing for directly measuring an amount of force or pressure being applied to the sensor. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the sensor is a piezoelectric sensor or piezoelectric crystal, the method further comprises the step of providing a resistor having a fixed resistance connected in series with the piezoelectric sensor. The method further provides for measuring a voltage drop across the fixed resistor, which voltage drop corresponds to an amount of change in force being applied to the piezoelectric sensor. An external voltage integration circuit converts the force change signal generated by the piezoelectric sensor into a signal proportional to the absolute value of the force being applied. The method may further provide for calculating a pressure being applied by the jaws from the measured amount of force being applied to the sensor. The method may further provide for visually displaying the calculated pressure. The method may further provide for the sounding of an audio alert corresponding to the amount of force or pressure being applied to the sensor.

According to yet another embodiment, a method for measuring an amount of force being applied by the jaws of a grasper is provided. The method comprises the step of providing a grasper comprising a handle and two jaws operably connected to the handle, which jaws can be actuated by the handle. The method further comprises the steps of providing a sensor located on or inside the handle and providing for indirectly measuring an amount of force or pressure being applied to the sensor at an actuator level. If the indirect measurement approach is used, a calibration procedure is implemented at manufacturing time to determine and store a calibration profile inside a non-volatile memory located in the grasper's handle which will be used to convert, in real-time, the indirect measurements taken into the force or pressure values applied at the jaws. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the sensor is a piezoelectric sensor or piezoelectric crystal, the method further comprises the step of providing a resistor having a fixed resistance connected in series with the piezoelectric sensor. The method further provides for measuring a voltage drop across the fixed resistor, which voltage drop corresponds to an amount of change in force being applied to the piezoelectric sensor. An external voltage integration circuit converts the force change signal generated by the piezoelectric sensor into a signal proportional to the absolute value of the force being applied. The method may further provide for calculating a pressure being applied by the jaws from the measured amount of force being applied to the sensor. The method may further provide for visually displaying the calculated pressure. The method may further provide for the sounding of an audio alert corresponding to the amount of force or pressure being applied to the sensor.

According to still another embodiment, a method for measuring an amount of force being applied by the jaws of a grasper for use in robotic surgery is provided. The method comprises the step of providing a grasper for use in robotic surgery, the grasper comprising a shaft and two jaws at a distal end of the shaft, which jaws can be actuated in response to a robot command. The method further comprises the steps of providing a sensor on an inner surface of one or both of the jaws, and providing for directly measuring an amount of pressure or force being applied to the sensor. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the sensor is a piezoelectric sensor or piezoelectric crystal, the method further comprises providing a resistor having a fixed resistance connected in series with the piezoelectric sensor. The method further provides for measuring a voltage drop across the fixed resistor, which voltage drop corresponds to an amount of change in force being applied to the piezoelectric sensor. An external voltage integration circuit converts the force change signal generated by the piezoelectric sensor into a signal proportional to the absolute value of the force being applied. A feedback can be provided to the robot of the measured voltage drop or the measured amount of force or pressure being applied to the sensor for use in adjusting the amount of force being applied by the jaws.

According to yet another embodiment, a method for measuring an amount of force being applied by the jaws of a grasper for use in robotic surgery is provided. The method comprises the step of providing a grasper for use in robotic surgery, the grasper comprising a shaft and two jaws at a distal end of the shaft, which jaws can be actuated in response to a robot command. The method further comprises the steps of providing a sensor at a proximal end of the shaft, at an actuator, or on a wrist of a robot arm, and providing for indirect measurement of the force or pressure being applied to the sensor at the actuator level. If the indirect measurement approach is used, a calibration procedure is implemented at manufacturing time to determine and store a calibration profile inside a non-volatile memory located remotely from the grasper's distant end of the shaft which will be used to convert, in real-time, the indirect measurements taken into the force or pressure values applied at the jaws. The sensor can be any type of pressure or force sensor, including but not limited to a piezoelectric sensor, a simple piezoelectric crystal, a Hall-Effect or a resistive strain gauge sensor, etc., all of which can be either stand-alone or integrated with signal-conditioning electronics (Wheatstone bridge, low-noise amplifier, A/D converter, etc.) into a single chip or single package sealed module. If the sensor is a piezoelectric sensor or piezoelectric crystal located on the grasper or the robot, the method further comprises providing a resistor having a fixed resistance connected in series with the piezoelectric sensor. The method further provides for measuring a voltage drop across the fixed resistor, which voltage drop corresponds to an amount of change in force being applied to the piezoelectric sensor. In this embodiment, a feedback can be provided to the robot of the measured voltage drop or the measured amount of force or pressure being applied to the sensor for use in adjusting the amount of force being applied by the jaws.

According to yet another embodiment, a surgical grasper comprises a shaft, two jaws at a distal end of the shaft, and a strain gauge sensor that uses the Hall Effect to measure a force being applied by the jaws. The strain gauge sensor can be integrated with signal-conditioning electronics (Wheatstone bridge, low-noise amplifier, A/D converter, etc.) into a single chip or a single package sealed module. A microprocessor and a non-volatile memory chip can be included for at least one of calibration parameter storage and forensic storage. A handle can be operably connected to the jaws, wherein the jaws can be actuated by the handle. The strain gauge sensor can be located on or inside the handle, on an inner surface of one or both of the jaws, or on or inside the shaft. The surgical grasper can be specifically designed for use in robotic surgery, wherein the jaws can be actuated in response to a robot command. In such a design, the strain gauge sensor can also be located at an actuator or on a wrist of a robot arm. The measured force can be fed back to the robot for use in adjusting the amount of force being applied by the jaws. A visual or audio signal can be provided corresponding to an amount of force being applied by the jaws.

According to still another embodiment, a surgical grasper comprises a shaft, two jaws at a distal end of the shaft, and a MEMS sensor. The MEMS sensor can be integrated with signal-conditioning electronics (Wheatstone bridge, low-noise amplifier, AID converter, etc.) into a single chip or a single package sealed module. A microprocessor and a non-volatile memory chip can be included for at least one of calibration parameter storage and forensic storage. A handle can be operably connected to the jaws, wherein the jaws can be actuated by the handle. The MEMS sensor can located on or inside the handle, on an inner surface of one or both of the jaws, or on or inside the shaft. The surgical grasper can be specifically designed for use in robotic surgery, wherein the jaws can be actuated in response to a robot command. In such a design, the MEMS sensor can also be located at an actuator or on a wrist of a robot arm. A measured value can be fed back to the robot for use in adjusting the amount of force being applied by the jaws. A visual or audio signal corresponding to an amount of force being applied by the jaws can also be provided.

According to yet another embodiment, a surgical feedback system comprises a surgical grasper capable of taking a force measurement, the grasper comprising a shaft, two jaws at a distal end of the shaft, and a sensor. A data concentrator is coupled to the grasper via a wired or wireless interface using a first data transmission protocol with internal storage, wherein the first data transmission protocol can be RS-232C, USB, Ethernet, Optical Fiber, Wireless USB, Wireless Ethernet, Firewire, Wi-Fi, 802.11B, 802.11g, Wi-Max, Wireless Telemetry or Bluetooth. In one embodiment, the data concentrator is wireless and the force measurement is transmitted via the first data transmission protocol at least once every 100 milliseconds to the data concentrator. The data concentrator can optionally multiplex a plurality of surgical graspers on a single data link to a monitoring station. The data concentrator can also be coupled to a visualizing display, a patient monitoring system, or a Hospital Information System via a wired or wireless interface using a second data transmission protocol for real-time and historical data transmissions from the surgical grasper. The visualizing display, the patient monitoring system, or the Hospital Information System can be Analog, DVI, HDMI, Ethernet, Wireless Telemetry, Wi-Fi, Wi-Max, TCP/IP, Web Service, or HL7. The data concentrator can store a history of the force measurements for up to a given time of continuous operation for forensic purposes.

According to still another embodiment, a method for measuring an amount of force being applied by the jaws of a grasper is provided. The method comprises the steps of providing a grasper comprising a shaft and two jaws, providing a strain gauge sensor, and providing for using the Hall Effect to measure an amount of force being applied to the strain gauge sensor. The strain gauge sensor can be integrated with signal-conditioning electronics (Wheatstone bridge, low-noise amplifier, A/D converter, etc.) into a single chip or a single package sealed module. The method can further comprise the steps of providing for calculating a pressure being applied by the jaws from the measured amount of force being applied to the strain gauge sensor, and providing for visually displaying the calculated pressure. The method can further comprise the step of providing for sounding an audio alert corresponding to an amount of force being applied to the strain gauge sensor. The method can further comprise the steps of providing a microprocessor and a non-volatile memory chip, providing for storing calibration parameters in the memory chip at manufacturing time, and providing for storing the history of time-stamped transmitted data in the memory chip over a useful life of the grasper. The method can still further comprise the step of providing a handle operably connected to the jaws, wherein the jaws can be actuated by the handle and the strain gauge sensor is provided on or inside the handle. The grasper can also be provided for use in robotic surgery, wherein the jaws can be actuated responsive to a robot command. The strain gauge sensor can be provided on or inside the shaft, on an inner surface of one or both of the jaws, at an actuator, or on a wrist of a robot arm. The method can further comprise the step of providing a feedback to the robot of the measured amount of force being applied to the strain gauge sensor for use in adjusting the amount of force being applied by the jaws.

According to yet another embodiment, a method for measuring an amount of force being applied by the jaws of a grasper is provided. The method comprises the steps of providing a grasper comprising a shaft and two jaws, and providing a MEMS sensor. The MEMS sensor can be integrated with signal-conditioning electronics (Wheatstone bridge, low-noise amplifier, A/D converter, etc.) into a single chip or a single package sealed module. The method can further comprise the steps of providing for calculating a pressure being applied by the jaws from the measured amount of force being applied to the MEMS sensor, and providing for visually displaying the calculated pressure. The method can further comprise the step of providing for sounding an audio alert corresponding to an amount of force being applied to the MEMS sensor. The method can still further comprise the steps of providing a microprocessor and a non-volatile memory chip, providing for storing calibration parameters in the memory chip at manufacturing time, and providing for storing the history of time-stamped transmitted data in the memory chip over a useful life of the grasper. The method can further comprise the step of providing a handle operably connected to the jaws, wherein the jaws can be actuated by the handle and the MEMS sensor is provided on or inside the handle. The grasper can be provided for use in robotic surgery, wherein the jaws can be actuated responsive to a robot command. The MEMS sensor can be provided on or inside the shaft, on an inner surface of one or both of the jaws, at an actuator, or on a wrist of a robot arm. The method can further comprise the step of providing a feedback to the robot of the measured amount of force being applied to the MEMS sensor for use in adjusting the amount of force being applied by the jaws.

According to still another embodiment, a method for obtaining surgical feedback is provided. The method comprises the step of providing a surgical grasper capable of taking a force measurement, wherein the grasper comprises a shaft, two jaws at a distal end of the shaft, and a sensor. The method also comprises the step of providing a data concentrator coupled to the grasper via a wired or wireless interface using a first data transmission protocol with internal storage, wherein the first data transmission protocol can be RS-232C, USB, Ethernet, Optical Fiber, Wireless USB, Wireless Ethernet, Firewire, Wi-Fi, 802.11B, 802.11g, Wi-Max, Wireless Telemetry or Bluetooth. In one embodiment, the data concentrator is wireless and the first data transmission protocol is capable of transmitting the force measurement to the data concentrator at least once every 100 milliseconds. The data concentrator can optionally be capable of multiplexing a plurality of surgical graspers on a single data link to a monitoring station. The method can further comprise the step of providing that the data concentrator is capable of being coupled to a visualizing display, a patient monitoring system, or a Hospital Information System via a wired or wireless interface and a second data transmission protocol for real-time and historical data transmissions from the surgical grasper. The visualizing display, the patient monitoring system, or the Hospital Information System can be Analog, DVI, HDMI, Ethernet, Wireless Telemetry, Wi-Fi, Wi-Max, TCP/IP, Web Service, or HL7. The data concentrator can be provided with the ability to store a history of the force measurements for up to a given time of continuous operation for forensic purposes.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a perspective view of a grasper in a surgical feedback system according to one embodiment of the present invention;

FIG. 2 is a schematic of a basic voltage divider circuit with no load;

FIG. 3 is a schematic of a circuit according to one embodiment of the present invention;

FIG. 4 is a perspective view of a portion of a grasper according to one embodiment of the present invention;

FIG. 5 is a perspective view of a portion of a grasper according to one embodiment of the present invention;

FIG. 6 is a perspective view of a portion of a grasper according to one embodiment of the present invention; and,

FIG. 7 is a perspective view of a grasper in a surgical feedback system according to one embodiment of the present invention.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. Particularly, the surgical instrument is described and shown herein as a grasper 10 for grasping and holding skin, soft tissue, muscle, fascia, arteries, veins, etc. during minimally-invasive surgery. However, it should be understood that the present invention may take the form of many different types of surgical instruments, for use in minimally-invasive surgeries or otherwise, used for grasping, holding, cutting, prodding, sewing, stitching, stapling, or pinching tissue or other bodily parts, including but not limited to open or endoscopic, pickups, graspers, cutters, scalpels, etc.

Gently handling tissue has long been a basic tenet for excellence in surgery, and the basic rationale of minimally-invasive surgery is to reduce trauma to the tissue. “Gentle touch” is an especially poignant skill in minimal feedback environments. Contemporary surgeons must learn surgical technique on simulators outside of the operating room, e.g., in “box trainers, virtual reality surgical simulators.” For this reason, the device and associated method of the present invention were created to teach and give feedback regarding a surgeon's gentle touch. Using this device and method, a surgeon's gentle touch can be detected, measured, and improved in a simple, intuitive, and cost-effective fashion, as the device of the present invention can be manufactured inexpensively while maintaining extremely high degrees of accuracy. Another application of the device and the associated method of the present invention is to provide real-time feedback to the surgeon during “live” minimally-invasive surgery, alerting the surgeon when predetermined programmed warning thresholds have been reached, while maintaining a complete record of the force and/or pressure applied during the surgery for forensic purposes.

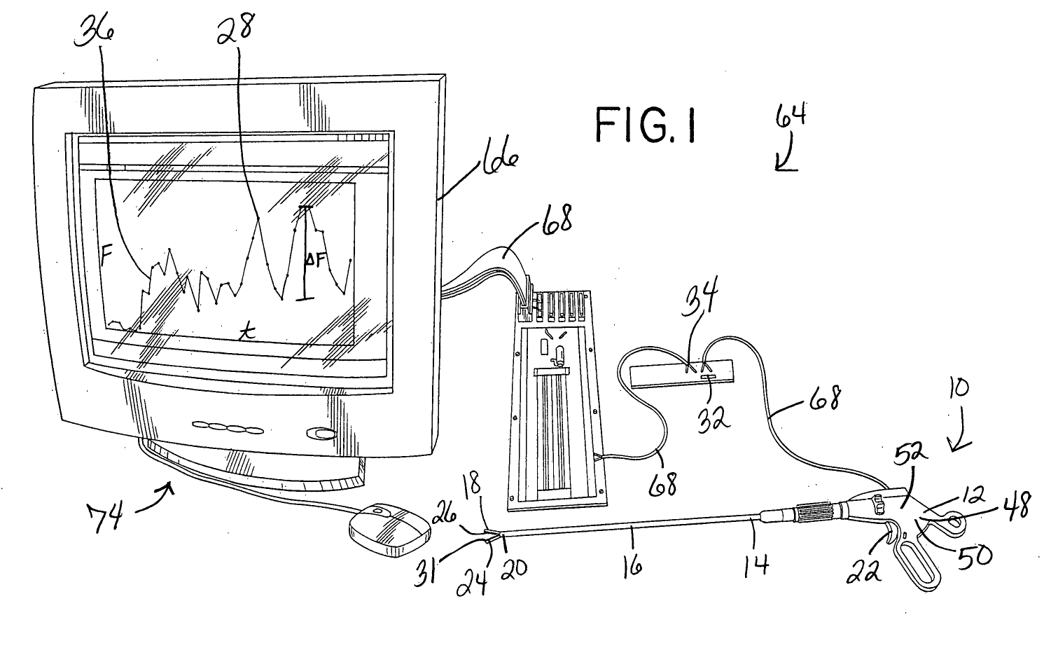

Referring now in detail to the FIGURES, and initially to FIG. 1, a surgical grasper 10 according to an embodiment of the present invention is shown. The grasper 10 comprises a handle 12 connected to a proximal end 14 of a shaft 16. There are grasping surfaces or jaws 18 at a distal end 20 of the shaft 16, which jaws 18 are operably connected to the handle 12 and can be actuated by pressing on a trigger 22 that is part of the handle 12. A sensor 24 is provided on the grasper 10. The sensor 24 can be located on an inner surface 26 of one or both of the jaws 18, allowing for direct measurement of an amount of pressure or force 28 being applied with the grasper 10. The sensor 24 can also be located on or inside the handle 12 or on or inside the shaft 16 for indirect measurement of the amount of pressure or force 28 being applied with the grasper 10 at an actuator level. If the indirect measurement approach is used, a calibration procedure is implemented at manufacturing time to determine and store a calibration profile inside a non-volatile memory 48 located in the handle 12 or elsewhere in the grasper 10, which will be used to convert, in real-time, the indirect measurements taken into the force or pressure values applied at the jaws 18 of the grasper 10.

A microprocessor 50 and the non-volatile memory 48 can be included for at least one of calibration parameter storage and forensic storage, i.e., to store a complete history of the force measured by the device during its use, up to a predetermined amount of time preferably equal to or greater than the “useful life” of the grasper 10 (most likely a single-use disposable). The calibration procedure can be used at manufacturing time to determine and store the calibration profile inside the non-volatile memory 48, which can be located anywhere on or in the device, including on or in the handle 12 or the shaft 16, and which will be used to convert, in real-time, the measurements taken into the pressure values applied at the jaws 18. A manufacturing calibration fixture (not shown) has a mechanical “finger” having a “width” that is mechanically and precisely adjustable in small increments (0.1 mm +/−5%) with a pressure sensor mounted on its active side and a computer-controlled “squeezer” that will apply pressure on the handle 12 mechanism until the pressure measured by the fixture equals the programmed value. The programmed value together with the “raw” pressure measured by the grasper's 10 remote sensor 24, which can be mounted inside the handle 12 or anywhere else on the grasper 10 that is feasible, is then recorded for storage in the non-volatile memory 48. The process is repeated until the entire range of pressures for which the grasper 10 is intended to function is covered. The process is again repeated for the entire range of pressures for each possible angle position of the grasper's 10 jaws 18 as determined by the handle's 12 ratcheting mechanism. The resulting 3-dimensional calibration table is then used by a microcomputer-based logic circuit 52 mounted inside the handle 12 or elsewhere in the grasper 10 to “look up” in real-time the pressure at the jaws 18 based on the pressure at the remote handle 12 mechanism or other remote actuator and the angle position of its ratcheting mechanism.

Any portion or all of the trigger 22, the handle 12, and/or the inner surface(s) 26 of the jaw(s) 18 can be substantially covered with the sensors 24. One sensor 24 can be as small as 1 mm or even smaller in many cases. The sensor 24 can be any type of force or pressure sensor, including but not limited to piezo, strain gauge, Hall Effect, electromechanical, variable capacitance, mechanical, MEMS, nanotechnology-enabled sensors, and any other known sensor 24 or combination of sensors 24 or sensing technology that can be used to measure an amount of force or pressure 28 or any other value that can be converted to a force or pressure value. Specific types of such sensors 24 include, but are not limited to, piezoelectric sensors 30, simple piezoelectric crystals 31, thin film 54, resistive strain gauge sensors 33, strain gauge sensors 56 that use the Hall Effect or a Wheatstone bridge, and MEMS 58 force or pressure sensors. The sensor 24 can also be a photosensor, such as a photoresistor or light-dependent resistor (LDR), an optical proximity sensor, or a fiber optic sensor, which work particularly well in embodiments where indirect measurements are taken at the actuator level, but all of which can be used in other embodiments as well. Numerous examples of sensors 24 that can be used in the present invention are described in JON S. WILSON, SENSOR TECHNOLOGY HANDBOOK (Newnes 2004). The sensor 24 can be either stand-alone or integrated with signal-conditioning electronics 35, such as a Wheatstone bridge, a low-noise amplifier, or an A/D converter, etc., into a single chip 60 or a single package sealed module 62.

Referring to FIGS. 1 thru 4, when the sensor 24 is the piezoelectric sensor 30 or the piezoelectric crystal 31, a resistor 32 having a fixed resistance is connected in series with the piezoelectric sensor 30 or crystal 31, wherein a voltage drop is measurable across the fixed resistor 32. The measured voltage drop corresponds to an amount of change in force ΔF being applied to the piezoelectric sensor 30 or crystal 31. A voltage integration circuit 34 converts the force change signal generated by the piezoelectric sensor 30 or crystal 31 into a signal proportional to the absolute value of the force being applied. This voltage integration circuit 34 is not necessary if the sensor 24 technology is based on a true pressure- or force-reading principle. A visual signal 36 and/or an audio signal can be provided corresponding to an amount of force being applied to the sensor 24.

Piezoelectric pressure sensors 30 use stacks of piezoelectric crystals 31 or ceramic elements (not shown) to convert the motion of a force-summing device to an electrical output. Piezoelectric sensors 30 and crystals 31 change resistance as their crystal structure is altered. In other words, the piezoelectric sensor's 30 or crystal's 31 resistance changes when force is applied or removed, i.e., when it is strained. The piezoresistive effect is the change in the bulk electrical resistivity that occurs when mechanical stress is applied to the piezoelectric sensor 30. It is preferable, but not required, that the resistance of the piezoelectric sensor 30 or crystal 31 drop as force is applied to its surface, so that a direct correlation can be drawn between the resistance level and the force being applied 28. Quartz, tourmaline, and several other naturally occurring piezoelectric crystals 31 are known to generate an electrical charge when strained, as are certain ceramics that are artificially polarized to be piezoelectric. Unlike strain gauge sensors 56, piezoelectric devices require no external excitation. However, due to their high impedance output and low signal levels, piezoelectric sensors 30 and crystals 31 do require signal-conditioning electronics 35. Because they are self-generating, piezoelectric sensors 30 and crystals 31 are dependent upon changes in pressure or strain to generate electrical charge, making them unsuitable for use with DC or steady-state. One advantage of piezoelectric sensors 30 and crystals 31 is their ruggedness, including the ability to perform accurately at high temperatures (without integral electronics). However, one skilled in the art understands the necessity of properly compensating piezoelectric devices in order to prevent possible shock, vibration, and/or variable sensitivity at different temperatures.

An insulating material (not shown) can be placed between the piezoelectric sensor 30 or crystal 31 and the jaw(s) 18, shaft 16, or other applicable part(s) of the grasper 10 to keep the circuit from grounding, as needed. This will depend on the material of which the jaws 18, shaft 16, or other applicable part(s) of the grasper 10 are made. While any known insulating material can be used, it is preferable that the insulating material can be easily sterilized, unless the grasper 10 is disposable.

For many types of piezoelectric sensors 30 and crystals 31, the resistance is extremely high when no force is being applied (essentially creating an open circuit) and extremely low when significant force is being applied (hundreds of ohms). This wide swing in resistance makes it difficult to measure the resistance change directly. The smaller the crystal lattice structure, the more difficult it is to measure the resistance change directly. The wide range of electrical signals and noise involved precludes the use of most widely available measurement equipment. Therefore, the resistor 32 with the fixed resistance is matched to and connected in series with the piezoelectric sensor 30 or crystal 31. The voltage drop is then measurable across the fixed resistor 32, which voltage drop corresponds to the amount of change in force ΔF being applied to the piezoelectric sensor 30 or crystal 31. By selecting the appropriate size of the fixed resistor 32 in series, the voltage drop is measurable for any piezoelectric sensor 30. The fixed resistor 32 must be matched to accommodate the range of the voltage drop required for the particular piezoelectric sensor 30 or crystal 31. A 2,000 ohm fixed resistor 32 was connected in series with the piezoelectric sensor 30 of FIG. 1 to facilitate measurement of the voltage drop.

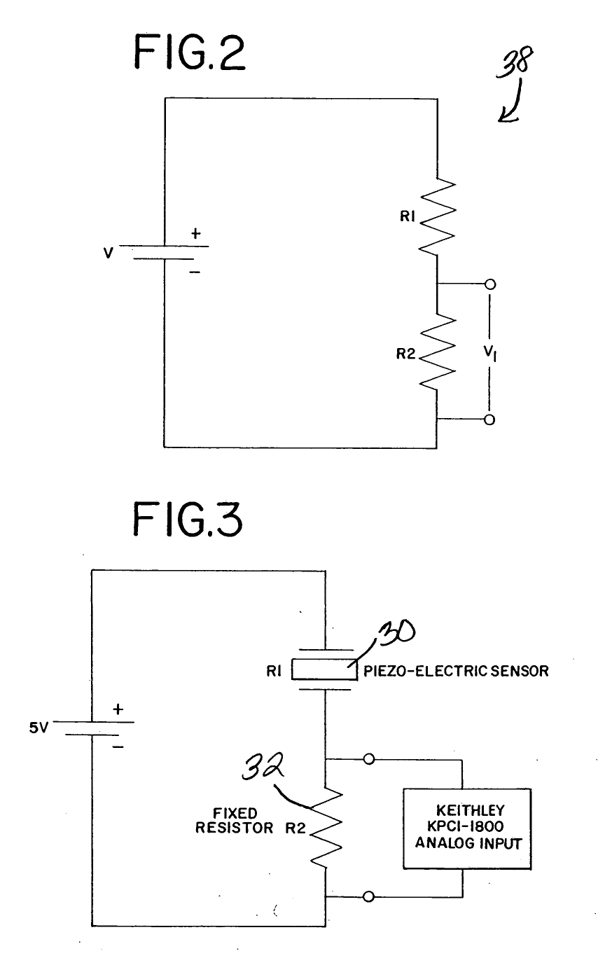

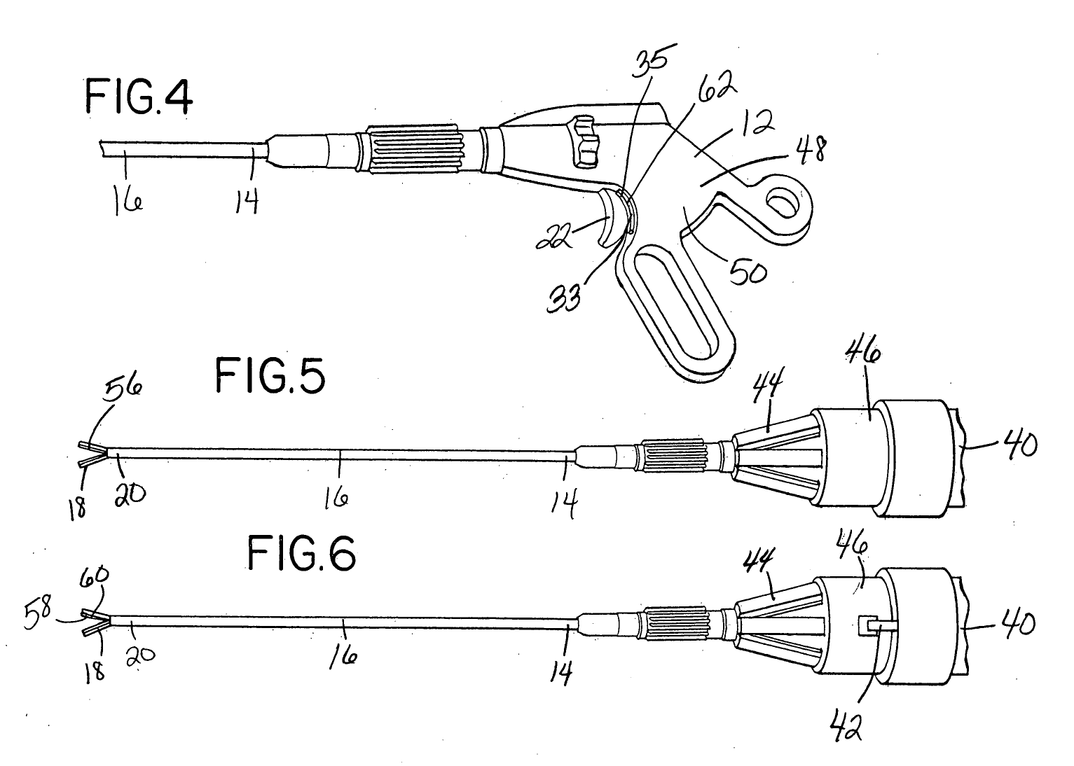

The basic building block for the piezoelectric sensor 30 or crystal 31 measurement device and method is a voltage divider 38. FIG. 2 illustrates a two-resistor R₁, R₂ voltage divider 38 with no load. Based on Ohm's law, the voltage across the fixed resistor R₂ can be determined using the following equation: Vₒᵤₜ=V₁(R₂/R₁+R₂). As is appreciated by one skilled in the art, once Vₒᵤₜ is determined, the current flowing through the circuit can be determined using the equation V=IR.

FIG. 3 illustrates one possible setup to measure the resulting current flowing through the piezoelectric sensor 30 or crystal 31 circuit. As the resistance R₁ of the piezoelectric sensor 30 or crystal 31 changes, the amount of current flowing through the circuit also changes. The piezoelectric sensor 30 and crystal 31 work like variable resistors, with R₁ varying in proportion to the amount of force 28 being applied to the piezoelectric sensor 30 or crystal 31. R₂ is fixed at the fixed resistor 32 and the voltage drop across it can be measured using a Keithley Instruments KPCI-1800 data acquisition board (board is Keithley Instruments Part No. KPCI-1801HC, used in conjunction with a dedicated screw terminal accessory Keithley Instruments Part No. STA-1800HC and shielded cable Keithley Instruments Part No. CAB-1802/S) running inside a PC with Microsoft Windows running Excel for data collection using a macro provided free with the board, or similar setup, as will be understood by those skilled in the art. The KPCI-1800 has an on-board 5V power supply to power the circuit. Since R₂ and V₁ are known and Vₒᵤₜ can be measured, the resistance of the piezoelectric sensor 30 and the circuit current can be determined using the preceding equations.

An executable file (.exe) was written using TestPoint V5.0 SN K141B-4350-019C with a start/stop function to graphically display in real-time the voltage outputs from the piezoelectric sensor 30 or crystal 31 in a strip chart type fashion. This gives a real-time reading similar to a ticker tape or an EKG machine. When force is applied to the piezoelectric sensor 30 or crystal 31, the voltage line goes up. Oppositely, when force on the piezoelectric sensor 30 or crystal 31 is lessened, the voltage line goes down accordingly. This gives the surgeon real-time feedback during “live” minimally-invasive surgery. Rather than displaying voltage output vs. time (not shown), the graphical display can display force vs. time (FIG. 1) or pressure vs. time (not shown). One skilled in the art will appreciate that converting the measured voltage outputs to force or pressure readings can be done using simple engineering calibrations and calculations. Various different types of equipment can be employed to measure the voltages and display those measurements. As will be understood by one skilled in the art, there are numerous ways, all within the scope of the present invention, to measure and display.

Referring to FIG. 5, strain gauge sensors 56 are also composed of materials that exhibit a significant change in bulk resistivity when strained, i.e., when force or pressure is applied (piezoresistive effect). Strain gauges 56 measure deformation due to pressure and usually comprise a long thin conductor (not shown), often printed onto a plastic backing in such a way that it occupies very little space. As the length of the conductor is altered, its cross-sectional area is also changed proportionally (Poisson Effect). The change in length and cross-sectional area causes an approximately proportional change in the resistance of the conductor. The change in resistance is largely proportional to both the change in length and the change in cross-sectional area. All strain gauge materials exhibit these properties, but the piezoresistive effect varies widely for different materials. For example, metal strain gauges exhibit relatively large piezoresistive effects, while silicon strain gauges are generally doped to resistivity levels that yield optimal thermoresistive and piezoresistive effects. The change in resistance is sometimes small and may require a reference resistance and other circuitry to compensate for other sources of resistance changes, such as temperature, as is understood by those skilled in the art. The strain gauges 56 can be bonded (glued), unbonded, sputtered, or of the semiconductor variety. Bonded discrete silicon strain gauge, diffused diaphragm, and sculptured diaphragm sensors are all viable options.

When the sensor 24 is the strain gauge sensor 56, the Hall Effect can be used to measure the force 28 being applied by the jaws 18. As pressure is added to the strain gauge 56, deformation occurs, which deformation causes a change in its electrical resistance. A current applied to the strain gauge 56 within a magnetic field creates the Hall effect and an EMF or “Hall voltage” is generated in a direction mutually perpendicular to directions of both the magnetic field and the current flow. The Hall voltage can then be detected with an instrumentation amplifier (DC excitation current) (not shown) or a lock-in amplifier (AC excitation current) (not shown). Alternatively, the electrical resistance change in the strain gauge sensor 56 can be measured using a Wheatstone bridge (not shown). As previously noted, the voltage integration circuit 34 is not necessary if the sensor 24 technology is based on a true pressure- or force-reading principle.

Another option, the variable capacitance sensor (not shown), has two plates, one of which is the diaphragm of the pressure sensor, which can be displaced relative to the other plate, causing the capacitance between the two plates to change. The change in capacitance can be used to vary an oscillator frequency or be detected by a bridge circuit. The measured capacitance corresponds to a force or pressure being applied to the sensor.

Still other options include photosensors or photoreflectors, including photoresistors or light-dependent resistors (LDR), optical proximity sensors, and fiber optic-enabled sensors. These work particularly well in embodiments where indirect measurements are taken at the actuator level, but can be used in other direct measurement embodiments, as well. Photosensors are electronic components that detect the presence of visible, infrared (IR), and/or ultraviolet (UV) light. Most photosensors consist of a photoconductive semiconductor for which the electrical conductance varies with the intensity of radiation striking the material. Common photosensors include photodiodes, bipolar phototransistors, and photosensitive field-effect transistors. These devices are similar to the ordinary diode, bipolar transistor, and field-effect transistor, respectively, with the addition of a transparent window to allow radiant energy to reach the junctions between the semiconductor materials inside.

Generally, optical proximity sensors require a light source, a detector and sensor control circuitry. The light source should generate light of a wavelength and frequency that the detector is able to detect and that is not likely to be generated by other nearby light sources. For this reason, IR light pulsed at a fixed frequency is a popular choice. The sensor control circuitry should be compatible with the pulsing frequency, as well. The detector can be a semiconductor device, such as a photodiode, which generates a small amount of electric current when light energy strikes it. The detector can also be a phototransistor or a photodarlington that allows current to flow when light strikes it. RetroHective-type photosensors package the light source and the detector in a single package for detecting targets that reflect light back to the receiver. Retroreflective-type photosensors are designed to recognize targets within a limited distance range only, and their output is proportional to the amount of light reflected back to the detector, thereby indicating the nearness of the target.

Phase modulation experienced by light traveling through an optical fiber exposed to external fields can be retrieved and processed using interferometry to determine a specific external field characteristic. When configured as an interferometer, an external disturbance that affects the length of the fiber, such as strain or pressure, causes a phase change in the light, which is relayed at high speeds through the optical fiber for detection. A Bragg grating can be used to detect variation in the fiber properties because when the fiber is illuminated with a light source, it will be reflected back from the grating section of the fiber. If a pressure or strain is applied to the grating section of the fiber, the grating period changes, as does the wavelength of the reflected light. The change in wavelength can be measured and converted to pressure or force values. Other fiber optic-enabled sensors can also be used to measure pressure or strain, such as an optical fiber with a Fabry-Perot cavity formed at its end. As pressure changes, deformation of the Fabry-Perot cavity diaphragm varies the cavity length. A light source illuminates the cavity, which reflects the light for detection by a spectrometer. Changes in the reflected light detected by the spectrometer are proportional to changes in the pressure. White light interferometry can be used to avoid error and noise caused by bending of the optical fiber and light source fluctuation. Additionally, some hybrid sensing systems use conventional sensor technology to obtain an electrical output, then convert the electrical output to an optical signal for transmission via an optical fiber.

Electronic pressure scanners combine miniature semiconductor strain gauges and solid-state electronic multiplexing into an integrated measurement system. A typical system includes a multiple transducer array, a low-level multiplexer, and an instrumentation amplifier in a shared housing. In such a system, each strain gauge is always measuring and its output is periodically sampled by the multiplexer.

Referring to FIG. 6 and following the general trend toward miniaturization of electronic components, pressure measurement devices have been produced that include the sensor itself plus associated electronic components needed to produce a useful output signal co-fabricated on a monolithic integrated circuit chip 60. Such a pressure measurement device is a type of microelectromechanical system (MEMS). Any of the sensors 24 described herein or otherwise available can be produced as MEMS sensors 58, as is understood by those skilled in the art. MEMS technology involves producing integrated micro devices or systems that combine electronic components formed on a semiconductor substrate (not shown) and three-dimensional mechanical components fabricated in the substrate. Sensors 24 and actuators are the primary categories of MEMS devices that have been developed and can range in size from micrometers to millimeters.

MEMS sensors 58 are fabricated using integrated circuit lithographic processing as well as micromachining. Micromachining processes include mask-based wet and dry etching and maskless processes such as focused ion beam etching, laser machining, ultrasonic drilling, and electrochemical discharge machining that produce high aspect-ratio features in the substrate. Some examples of MEMS sensors 58 for pressure measurement are disclosed in M. Schuenemann, et al., “A Highly Flexible Design and Production Framework for Modularized Microelectromechanical Systems,” Micromachine Devices, Vol. 3, March 1998. When the MEMS sensor 58 is used, the voltage integration circuit 34 may not be necessary, for example, if the sensor technology is based on a true pressure- or force-reading principle. This will naturally depend on the type of MEMS sensor 58 used.

Even smaller than MEMS sensors 58 are nanotechnology-enabled sensors or nanosensors. Current nanotechnology permits operation on the scale of atoms and molecules. Benefits due to the reduced size of nanosensors include decreased weight, decreased power requirements and increased sensitivity. There are many different types of nanosensors, some of which are manufactured using the conventional methods of lithography, etching and deposition, and others that are built using individual atoms and molecules. For instance, nanotubes, which are narrow hollow cylinders formed of carbon atoms, can be grown on existing structures. Nanotubes can be used to sense pressure and strain because the orientation of the carbon atoms directly affects its conducting and semi-conducting properties. Existing integrated circuit technologies can be used to add nanosensors to integrated electronic circuits, and chips including nanosensors can be used as building blocks to make more complex sensors. It is understood by those skilled in the art that MEMS and nanotechnology can be combined to develop hybrid sensor systems. Nanosensors are generally very sensitive and prone to degradation from the presence of foreign substances and extreme temperatures, the effects of which become more significant on the nano-scale. Such degradation can be counteracted by installing hundreds of nanosensors in a small space, which allows malfunctioning sensors to be ignored in favor of properly functioning sensors.

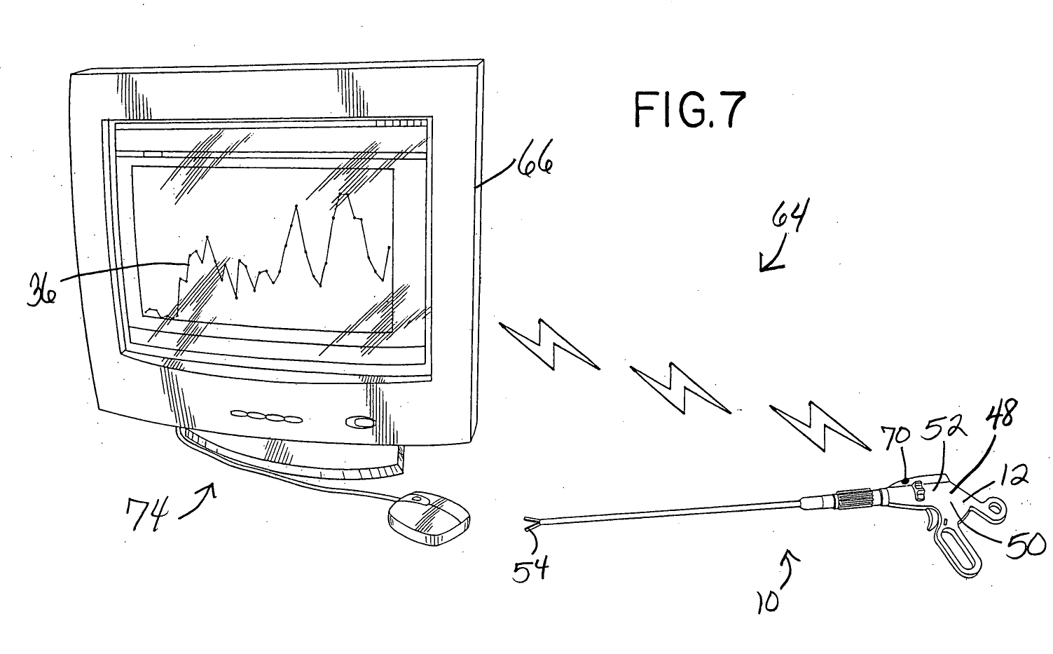

An audio alert and/or a visual display or signal 36 corresponding to the amount of force 28 being applied to the sensor 24 can be provided. The audio alert and/or the visual display or signal 36 can be used to provide real-time feedback to the surgeon during “live” minimally-invasive surgery, and can also be used to alert the surgeon when predetermined programmed warning thresholds are reached. The audio alert can be any type of audio alert, including but not limited to tones that get louder or faster or both as force is increased. The visual display or signal 36 can be any type of visual display or signal 36, including but not limited to a graphic display (FIGS. 1 and 7), a changing numerical display, or actual or virtual lights (green, yellow, red) to indicate how much force you are applying, i.e., red means “too much,” yellow means “you are approaching too much,” and green is “safe.” In this way, the sensor 24 works like the tactile sensors in the surgeon's fingertips, giving the surgeon feedback regarding the amount of force 28 being applied.

As there is always the risk of subsequent damage to the components of the present invention through incorrect sterilization, a single-use disposable grasper 10 is preferable, wherein the grasper 10 is tested in manufacturing, sterilized and packed to retain sterilization. There are four basic types of sterilization that are used in the manufacturing of medical devices: (1) ethylene oxide (EtO) sterilization (chemical gas)—good choice for most devices containing electronics, but only if the electronics are sealed in a plastic housing so as to not be directly exposed to the chemical gas; (2) steam sterilization (temperature/pressure strain)—not generally a good choice for devices containing electronics; (3) gamma radiation—also not generally a good choice for devices containing electronics; and, (4) electron-beam radiation (can be directed very precisely to sterilizing just portions, if needed)—considered less “harsh” than gamma radiation, but may need to be tested on the particular sensor 24 being used. If the sensor 24 is chip-based, meaning that the sensor 24 is integrated with the signal-conditioning electronics 35 and the whole circuit is encapsulated in a plastic or flexible rubber housing by the manufacturer, EtO sterilization is preferred. EtO sterilization is also preferred for piezoelectric sensors 30, crystals 31 or resistive strain gauge sensors 33 combined with electronics and encapsulated in plastic or rubber. However, if the piezoelectric sensor 30, crystal 31 or resistive strain gauge sensor 33 is not encapsulated or otherwise sealed, electron-beam sterilization may be preferred.

The graspers 10 according to the present invention can also be manufactured as two-part instruments—with a first part being a permanent portion and a second part being a disposable portion. In such an embodiment, it is preferable that the handle 12 is part of the permanent portion and the jaws 18 are part of the disposable portion. Of course, other configurations are possible as well. There are also non-disposable graspers 10 according to the present invention that may or may not need to be taken apart to be sterilized, depending on the particular design, as is understood by those skilled in the art. An advantage of the piezoelectric sensor 30, the simple piezoelectric crystal 31, and the resistive strain gauge sensor 33 is that they can be easily sterilized using standard hospital sterilization equipment. For example, autoclaving can be used, depending on the peak temperature, as is understood by those skilled in the art. Other methods of sterilizing like immersion in/pulverization with a liquid “germicide” followed by an adequate drying cycle in a sterile chamber are also possible, if the electronics can be tightly sealed in an injection-molded plastic shroud or otherwise sealed to prevent liquid ingress.

This is an improvement over a prior art attempt to use mechanical drums to sense force. Mechanical drums cannot be easily sterilized without taking the entire mechanism apart, so as to protect its many small mechanical moving parts. This is unworkable in an operating room environment where small parts could be easily lost and instruments need to be sterilized quickly for use on the next patient. The present invention is also an improvement over complicated prior art instruments that use ultrasound, high energy current, and vibration, along with software to “sense action,” because the device and method of the present invention provide for direct measurement of force and/or pressure.

Referring to FIGS. 5 and 6, according to another embodiment of the invention, a surgical grasper 10 is specifically designed for use in robotic surgery. The grasper 10 comprises a shaft 16, two jaws 18 located at a distal end 20 of the shaft 16, and a sensor 24. The jaws 18 can be actuated in response to a robot 40 command. The sensor 24 can be located anywhere on or in the grasper 10 or on or in the robot 40, including on an inner surface 26 of one or both of the jaws 18 for direct measurement of the amount of pressure or force 28 being applied with the grasper 10. The sensor 24 can also be located at a proximal end 14 of the shaft 16 or anywhere on or in the shaft 16, at an actuator 42, or on a wrist 44 of a robot arm 46 for indirect measurement of the amount of pressure or force 28 being applied with the grasper 10 at the actuator level. If the indirect measurement approach is used, a calibration procedure is implemented at manufacturing time to determine and store a calibration profile inside a non-volatile memory 48 located remotely from the distal end 20 of the shaft 16 which will be used to convert, in real-time, the indirect measurements taken into the force or pressure values applied at the jaws 18.

A microprocessor 50 and the non-volatile memory 48 can be included for at least one of calibration parameter storage and forensic storage, i.e., to store a complete history of the force measured by the device during its use, up to a predetermined amount of time preferably equal to or greater than the “useful life” of the grasper 10 (most likely a single-use disposable). The calibration procedure can be used at manufacturing time to determine and store the calibration profile inside the non-volatile memory 48, which can be located anywhere on or in the device, including on or in the handle 12 or the shaft 16, and which will be used to convert, in real-time, the measurements taken into the pressure values applied at the jaws 18. A manufacturing calibration fixture (not shown) has a mechanical “finger” having a “width” that is mechanically and precisely adjustable in small increments (0.1 mm +/−5%) with a pressure sensor mounted on its active side and a computer-controlled “squeezer” that will apply pressure on the grasper's handle actuator until the pressure measured by the fixture equals the programmed value. The programmed value together with the “raw” pressure measured by the grasper's remote sensor 24, which can be mounted inside the handle 12 or anywhere else on the grasper 10 that is feasible, is then recorded for storage in the non-volatile memory 48. The process is repeated until the entire range of pressures for which the grasper 10 is intended to function is covered. The process is again repeated for the entire range of pressures for each possible angle position of the jaws 18 as determined by the handle's 12 ratcheting mechanism. The resulting 3-dimensional calibration table is then used by a microcomputer-based logic circuit 52 mounted inside the handle 12 or elsewhere in the grasper 10 to “look up” in real-time the pressure at the jaws 18 based on the pressure at the handle mechanism or other remote actuator and the angle position of the ratchet mechanism.

The sensor 24 can be any type of force or pressure sensor, including but not limited to piezo, strain gauge, Hall Effect, electromechanical, variable capacitance, mechanical, MEMS, nanotechnology-enabled sensors, and any other known sensor 24 or combination of sensors 24 or sensing technology that can be used to measure force or pressure or any other value that can be converted to a force or pressure value. Specific types of such sensors 24 include, but are not limited to, piezoelectric sensors 30, simple piezoelectric crystals 31, thin film 54, resistive strain gauge sensors 33, strain gauge sensors 56 that use the Hall Effect or a Wheatstone bridge, and MEMS 58 force or pressure sensors. The sensor 24 can also be a photosensor, such as a photoresistor or light-dependent resistor (LDR), an optical proximity sensor, or a fiber optic sensor, which work particularly well in embodiments where indirect measurements are taken at the actuator level, but all of which can be used in other embodiments as well. Numerous examples of sensors 24 that can be used in the present invention are described in JON S. WILSON, SENSOR TECHNOLOGY HANDBOOK (Newnes 2004). The sensor 24 can be either stand-alone or integrated with signal-conditioning electronics 35, such as a Wheatstone bridge, a low-noise amplifier, or an A/D converter, etc., into a single chip 60 or single package sealed module 62.

When the sensor 24 is the piezoelectric sensor 30 or the piezoelectric crystal 31, a resistor 32 having a fixed resistance is connected in series with the piezoelectric sensor 30 or crystal 31, wherein a voltage drop is measurable across the fixed resistor 32. The measured voltage drop corresponds to an amount of change in force ΔF being applied to the piezoelectric sensor 30 or crystal 31. A voltage integration circuit 34 converts the force change signal generated by the piezoelectric sensor 30 or crystal 31 into a signal proportional to the absolute value of the force being applied. As previously noted, this voltage integration circuit 34 is not necessary if the sensor 24 technology is based on a true pressure- or force-reading principle. In this embodiment, the processed voltage or the raw measured voltage drop can be fed back to the robot 40 for use in adjusting the amount of force 28 being applied by the jaws 18. A visual signal 36 and/or an audio signal can be provided corresponding to an amount of force or pressure being applied to the sensor 24.

When the sensor 24 is the strain gauge sensor 56, the Hall Effect can be used to measure the force being applied by the jaws 18. As pressure is added to the strain gauge 56, deformation occurs, which deformation causes a change in its electrical resistance. A current applied to the strain gauge 56 within a magnetic field creates the Hall effect and an EMF or “Hall voltage” is generated in a direction mutually perpendicular to the directions of both the magnetic field and the current flow. The Hall voltage can then be detected with an instrumentation amplifier (DC excitation current) (not shown) or a lock-in amplifier (AC excitation current) (not shown). Alternatively, the electrical resistance change in the strain gauge sensor 56 can be measured using a Wheatstone bridge. As previously noted, the voltage integration circuit 34 is not necessary if the sensor 24 technology is based on a true pressure- or force-reading principle.

When the sensor 24 is the MEMS sensor 58, the voltage integration circuit 34 is not necessary if the sensor 24 technology is based on a true pressure- or force-reading principle. This will naturally depend on the type of MEMS sensor 58 used.

Referring to FIGS. 1 and 7, according to another embodiment of the present invention, a surgical feedback system 64 comprises a surgical grasper 10 capable of taking a force or pressure measurement 28 and a data concentrator 66 coupled to the grasper 10 via a wired or wireless interface using a first data transmission protocol with internal storage. The grasper 10 comprises a shaft 16, two jaws 18 at a distal end 20 of the shaft 16, and a sensor 24. As noted above, the sensor 24 can be any type of force or pressure sensor, including but not limited to piezo, strain gauge, Hall Effect, electromechanical, variable capacitance, mechanical, MEMS, nanotechnology-enabled sensors, and any other known sensor 24 or combination of sensors 24 or sensing technology that can be used to measure force or pressure or any other value that can be converted to a force or pressure value. Specific types of such sensors 24 include, but are not limited to, piezoelectric sensors 30, simple piezoelectric crystals 31, thin film 54, resistive strain gauge sensors 33, strain gauge sensors 56 that use the Hall Effect or a Wheatstone bridge, and MEMS 58 force or pressure sensors. The sensor 24 can also be a photosensor, such as a photoresistor or light-dependent resistor (LDR), an optical proximity sensor, or a fiber optic sensor, which work particularly well in embodiments where indirect measurements are taken at the actuator level, but all of which can be used in other embodiments as well. Numerous examples of sensors 24 that can be used in the present invention are described in JON S. WILSON, SENSOR TECHNOLOGY HANDBOOK (Newnes 2004). The sensor 24 can be either stand-alone or integrated with signal-conditioning electronics 35, such as a Wheatstone bridge, a low-noise amplifier, or an A/D converter, etc., into a single chip 60 or a single package sealed module 62.

The first data transmission protocol can be any type of data transmission protocol, off-the-shelf or otherwise, including RS-232C, USB, Ethernet, Optical Fiber, Wireless USB, Wireless Ethernet, Firewire, Wi-Fi, 802.11B, 802.11g, Wi-Max, Wireless Telemetry or Bluetooth. Each individual grasper's 10 logic circuit 52 mounted inside its handle 12 or elsewhere in the grasper 10 communicates the processed digital force or pressure measurement in real-time either through a wired data cable 68 connected to its handle 12 or elsewhere on the grasper 10 using a standard communication protocol (RS-232C, USB, Ethernet, etc.) or through a wireless transceiver 70 also mounted inside the handle 12 or elsewhere in the grasper 10 (Bluetooth, 802.11B, 802.11g, Wi-Max, Wireless Telemetry, etc.). The wireless transceiver 70 (i.e., using low-power Bluetooth) transmits the force or pressure measurement repeatedly, i.e., every 100 milliseconds, to a wireless data concentrator 66. In the shown embodiments, the data concentrator 66 is wireless and the force measurement is transmitted via the first data transmission protocol at least once every 100 milliseconds to the data concentrator 66 built into a computer 74.

The wired or wireless data concentrator 66 can optionally receive, store, and multiplex wired or wireless real-time data feeds from a plurality of individual graspers 10 (usually not more than 15, but can be any number) on a single data link, either wired or wireless, for retransmission to a patient monitoring station and/or system (wired or wireless) (not shown), an analog or digital video display (full screen or mixed video inset) (not shown), and/or a Hospital Information System (HIS) interface (HL7 protocol, RS-232, RS-422, Ethernet, etc.) (not shown), all of which can be off-the-shelf or otherwise built to suit specific needs. The data concentrator 66 is coupled to the visualizing display, the monitoring station or system, or the HIS via a wired or wireless interface using a second data transmission protocol, as required by the system, for real-time and historical data transmission from the connected surgical grasper(s) 10. The visualizing display, the patient monitoring system, and the Hospital Information System can be of any type, including Analog, DVI, HDMI, Ethernet, Wireless Telemetry, Wi-Fi, Wi-Max, TCP/IP, Web Service, or HL7. The data concentrator 66 can store a history of the force measurements for up to a given time of continuous operation for forensic purposes. The data concentrator 66 preferably stores up to two days' worth of data for each channel (each grasper 10 has its own channel) as a buffer against temporary communication loss and for later forensic investigation purposes (the “black-box” concept).

According to another embodiment of the present invention, a method for measuring an amount of force or pressure 28 being applied by the jaws 18 of a grasper 10 is provided. The method comprises the step of providing the grasper 10 comprising a handle 12 and two jaws 18 operably connected to the handle 12, which jaws 18 can be actuated by the handle 12. The method further comprises the steps of providing a sensor 24 on the grasper 10, and providing for measuring the amount of force or pressure 28 being applied to the sensor 24. The sensor 24 can be provided anywhere on the grasper 10, including on an inner surface 26 of one or both of the jaws 18 for direct measurement of the amount of pressure or force 28 being applied with the grasper 10. The sensor 24 can also be provided on or inside the handle 12 for indirect measurement of the amount of pressure or force 28 being applied with the grasper 10 at an actuator level. If the indirect measurement approach is used, a calibration procedure is implemented at manufacturing time to determine and store a calibration profile inside a non-volatile memory 48 located in the grasper's handle 12 which will be used to convert, in real-time, the indirect measurements taken into the force or pressure values applied at the jaws 18.

The method optionally comprises the steps of providing for calculating a pressure being applied by the jaws 18 from the measured amount of force 28 being applied to the sensor 24, and providing for visually displaying the calculated pressure, and vice-versa. The method optionally comprises the step of providing for the sounding of an audio alert corresponding to the amount of force being applied to the sensor 24. The sensor 24 can be any type of force or pressure sensor, including but not limited to piezo, strain gauge, Hall Effect, electromechanical, variable capacitance, mechanical, MEMS, nanotechnology-enabled sensors, and any other known sensor 24 or combination of sensors 24 or sensing technology that can be used to measure force or pressure or any other value that can be converted to a force or pressure value. Specific types of such sensors 24 include, but are not limited to, piezoelectric sensors 30, simple piezoelectric crystals 31, thin film 54, resistive strain gauge sensors 33, strain gauge sensors 56 that use the Hall Effect or a Wheatstone bridge, and MEMS 58 force or pressure sensors. The sensor 24 can also be a photosensor, such as a photoresistor or light-dependent resistor (LDR), an optical proximity sensor, or a fiber optic sensor, which work particularly well in embodiments where indirect measurements are taken at the actuator level, but all of which can be used in other embodiments, as well. Numerous examples of sensors 24 that can be used in the present invention are described in JON S. WILSON, SENSOR TECHNOLOGY HANDBOOK (Newnes 2004). The sensor 24 can be either stand-alone or integrated with signal-conditioning electronics 35, such as a Wheatstone bridge, a low-noise amplifier, or an A/D converter, etc., into a single chip 60 or a single package sealed module 62.

When the sensor 24 is a piezoelectric sensor 30 or piezoelectric crystal 31, the method further comprises the steps of providing a resistor 32 having a fixed resistance connected in series with the piezoelectric sensor 30 or crystal 31 and measuring a voltage drop across the fixed resistor 32, which voltage drop corresponds to an amount of change in force ΔF being applied to the piezoelectric sensor 30 or crystal 31. An external voltage integration circuit 34 converts the force change signal generated by the piezoelectric sensor 30 or crystal 31 into a signal proportional to the absolute value of the force being applied. As previously noted, this voltage integration circuit 34 is not necessary if the sensor 24 technology is based on a true pressure- or force-reading principle.

According to another embodiment of the present invention, a method for measuring an amount of force or pressure 28 being applied by the jaws 18 of a grasper 10 for use in robotic surgery is provided. The method comprises the step of providing the grasper 10 for use in robotic surgery, the grasper 10 comprising a shaft 16 and two jaws 18 at a distal end 20 of the shaft 16, which jaws 18 can be actuated responsive to a robot 40 command. The method further comprises the steps of providing a sensor 24, and providing for measuring the amount of force or pressure 28 being applied to the sensor 24. The sensor 24 can be any type of force or pressure sensor, including but not limited to piezo, strain gauge, Hall Effect, electromechanical, variable capacitance, mechanical, MEMS, nanotechnology-enabled sensors, and any other known sensor 24 or combination of sensors 24 or sensing technology that can be used to measure force or pressure or any other value that can be converted to a force or pressure value. Specific types of such sensors 24 include, but are not limited to, piezoelectric sensors 30, simple piezoelectric crystals 31, thin film 54, resistive strain gauge sensors 33, strain gauge sensors 56 that use the Hall Effect or a Wheatstone bridge, and MEMS 58 force or pressure sensors. The sensor 24 can also be a photosensor, such as a photoresistor or light-dependent resistor (LDR), an optical proximity sensor, or a fiber optic sensor, which work particularly well in embodiments where indirect measurements are taken at the actuator level, but all of which can be used in other embodiments, as well. Numerous examples of sensors 24 that can be used in the present invention are described in JON S. WILSON, SENSOR TECHNOLOGY HANDBOOK (Newnes 2004). The sensor 24 can be either stand-alone or integrated with signal-conditioning electronics 35, such as a Wheatstone bridge, a low-noise amplifier, or an AID converter, etc., into a single chip 60 or a single package sealed module 62.

When the sensor 24 is a piezoelectric sensor 30 or piezoelectric crystal 31, the method further comprises the steps of providing a resistor 32 having a fixed resistance connected in series with the piezoelectric sensor 30 or crystal 31, and measuring a voltage drop across the fixed resistor 32, which voltage drop corresponds to an amount of change in force ΔF being applied to the piezoelectric sensor 30 or crystal 31.

The sensor 24 can be provided anywhere on the grasper 10 or the robot 40, including on an inner surface 26 of one or both of the jaws 18 for direct measurement of the amount of pressure or force 28 being applied with the grasper 10. The sensor 24 can also be provided at a proximal end 14 of the shaft 16 or anywhere on the shaft 16, at an actuator 42, or on a wrist 44 of a robot arm 46 for indirect measurement of the amount of pressure or force 28 being applied with the grasper 10 at the actuator level. If the indirect measurement approach is used, a calibration procedure is implemented at manufacturing time to determine and store a calibration profile inside a non-volatile memory 48 located remotely from the distal end 20 of the shaft 16, which will be used to convert, in real-time, the indirect measurements taken into the force or pressure values applied at the jaws 18. An external voltage integration circuit 34 converts the force change signal generated by the piezoelectric sensor 30 or crystal 31 into a signal proportional to the absolute value of the force being applied. As previously noted, this voltage integration circuit 34 is not necessary if the sensor 24 technology is based on a true pressure- or force-reading principle. The method further comprises providing a feedback to the robot 40 of the measured amount of force or pressure 28 being applied to the sensor 24 or the raw measured voltage drop for use in adjusting the amount of force or pressure 28 being applied by the jaws 18 of the grasper 10.

According to another embodiment of the present invention, a method for measuring an amount of force 28 being applied by the jaws 18 of a grasper 10 comprises the steps of providing a grasper 10, providing a strain gauge sensor 56, and providing for using the Hall Effect to measure an amount of force 28 being applied to the strain gauge sensor 56. The grasper 10 comprises a shaft 16 and two jaws 18. The strain gauge sensor 56 can be integrated with signal-conditioning electronics 35 into a single chip 60 or a single package sealed module 62. The method can further comprise the steps of providing for calculating a pressure being applied by the jaws 18 from the measured amount of force 28 being applied to the strain gauge sensor 56, and providing for visually displaying the calculated pressure. The method can further comprise the step of providing for sounding an audio alert corresponding to an amount of force being applied to the strain gauge sensor 56. The method can further comprise the steps of providing a microprocessor 50 and a non-volatile memory chip 48, providing for storing calibration parameters in the memory chip 48 at manufacturing time, and providing for storing the history of time-stamped transmitted data in the memory chip 48 over a useful life of the grasper 10. The method can still further comprise the step of providing a handle 12 operably connected to the jaws 18, wherein the jaws 18 can be actuated by the handle 12 ,and the strain gauge sensor 56 can be provided on or inside the handle 12, on an inner surface 26 of one or both of the jaws 18, or on or in the shaft 16. The grasper 10 can be specifically provided for use in robotic surgery, wherein the jaws 18 can be actuated responsive to a robot 40 command, and the strain gauge sensor 56 can be provided on or inside the shaft 16, on an inner surface 26 of one or both of the jaws 18, at an actuator 42, or on a wrist 44 of a robot arm 46. The method can further comprise the step of providing a feedback to the robot 40 of the measured amount of force 28 being applied to the strain gauge sensor 56 for use in adjusting the amount of force being applied by the jaws 18.

According to another embodiment of the present invention, a method for measuring an amount of force 28 being applied by the jaws 18 of a grasper 10 comprises the steps of providing a grasper 10 comprising a shaft 16 and two jaws 18, and providing a MEMS sensor 58. The MEMS sensor 58 can be integrated with signal-conditioning electronics 35 into a single chip 60 or a single package sealed module 62. The method further comprises the steps of providing for calculating a pressure being applied by the jaws 18 from the measured amount of force 28 being applied to the MEMS sensor 58, and providing for visually displaying the calculated pressure. The method further comprises the step of providing for sounding an audio alert corresponding to an amount of force being applied to the MEMS sensor 58. The method further comprises the steps of providing a microprocessor 50 and a non-volatile memory chip 48, providing for storing calibration parameters in the memory chip 48 at manufacturing time, and providing for storing the history of time-stamped transmitted data in the memory chip 48 over a useful life of the grasper 10. The method further comprises the step of providing a handle 12 operably connected to the jaws 18, wherein the jaws 18 can be actuated by the handle 12 and the MEMS sensor 58 can be provided on or inside the handle 12, on an inner surface of one or both of the jaws 18, or on or in the shaft 16. The grasper 10 can be specifically provided for use in robotic surgery, wherein the jaws 18 can be actuated responsive to a robot 40 command and the MEMS sensor 58 can be provided on or inside the shaft 16, on an inner surface 26 of one or both of the jaws 18, at an actuator 42, or on a wrist 44 of a robot arm 46. The method further comprises the step of providing a feedback to the robot 40 of the measured amount of force 28 being applied to the MEMS sensor 58 for use in adjusting the amount of force being applied by the jaws 18.

According to another embodiment of the present invention, a method for obtaining surgical feedback 64 comprises the steps of providing a surgical grasper 10 capable of taking a force measurement 28, and providing a data concentrator 66 coupled to the grasper 10 via a wired or wireless interface using a first data transmission protocol with internal storage. The grasper 10 comprises a shaft 16, two jaws 18 at a distal end 20 of the shaft 16, and a sensor 24. As noted above, the sensor 24 can be any type of force or pressure sensor, including but not limited to piezo, strain gauge, Hall Effect, electromechanical, variable capacitance, mechanical, MEMS, nanotechnology-enabled sensors, and any other known sensor 24 or combination of sensors 24 or sensing technology that can be used to measure force or pressure or any other value that can be converted to a force or pressure value. Specific types of such sensors 24 include, but are not limited to, piezoelectric sensors 30, simple piezoelectric crystals 31, thin film 54, resistive strain gauge sensors 33, strain gauge sensors 56 that use the Hall Effect or a Wheatstone bridge, and MEMS 58 force or pressure sensors. The sensor 24 can also be a photosensor, such as a photoresistor or light-dependent resistor (LDR), an optical proximity sensor, or a fiber optic sensor, which work particularly well in embodiments where indirect measurements are taken at the actuator level, but all of which can be used in other embodiments as well. Numerous examples of sensors 24 that can be used in the present invention are described in JON S. WILSON, SENSOR TECHNOLOGY HANDBOOK (Newnes 2004). The sensor 24 can be either stand-alone or integrated with signal-conditioning electronics 35, such as a Wheatstone bridge, a low-noise amplifier, or an A/D converter, etc., into a single chip 60 or single package sealed module 62.

The first data transmission protocol can be any type of data transmission protocol, including RS-232C, USB, Ethernet, Optical Fiber, Wireless USB, Wireless Ethernet, Firewire, Wi-Fi, 802.11B, 802.11g, Wi-Max, Wireless Telemetry and Bluetooth. In one embodiment, the data concentrator 66 is wireless and the first data transmission protocol is capable of transmitting the force measurement 28 to the data concentrator at least once every 100 milliseconds. The method further comprises the step of providing that the data concentrator 66 is capable of being coupled to a visualizing display, a patient monitoring system or station (not shown), or a Hospital Information System (not shown) via a wired or wireless interface and a second data transmission protocol for real-time and historical data transmissions from the surgical grasper 10. The visualizing display, the patient monitoring system, and the Hospital Information System can be of any type, including Analog, DVI, HDMI, Ethernet, Wireless Telemetry, Wi-Fi, Wi-Max, TCP/IP, Web Service, and HL7. The data concentrator 66 can be provided as capable of multiplexing a plurality of surgical graspers 10 on a single data link to the patient monitoring station or system (not shown). The data concentrator 66 stores a history of the force measurements 28 for up to a given time of continuous operation of the grasper 10 for forensic purposes.

Additionally, it should be understood that the present invention is applicable to any minimal feedback environment, including but not limited to use in minimally-invasive surgery, to provide real-time feedback to the surgeon during the surgery, alerting the surgeon when predetermined programmed warning thresholds have been reached while maintaining a complete record of the force and/or pressure applied during the surgery in the non-volatile memory 48 located within the device for forensic purposes. The present invention is also intended to be used in box trainers (not shown) or virtual reality surgical simulators (not shown) for training residents to be surgeons. Specifically, the sensors 24 can be placed on either the teaching surgical instruments or on the practice organs or both. Then, the instructing surgeon has an objective way via the audio alert and/or the visual display signal 36 to determine whether the resident is squeezing enough or squeezing too much.

Several alternative embodiments and examples have been described and illustrated herein. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. A person of ordinary skill in the art would also appreciate that, as Pressure=Force/Area, a simple calculation can be used to switch between pressure and force. Therefore, whenever it makes sense to do so, anytime force is mentioned herein, this invention should be understood to also apply to pressure. Similarly, whenever it makes sense to do so, anytime pressure is mentioned herein, this invention should be understood to also apply to force. Additionally, the terms “1,” “2,” “first,” “second,” “primary,” “secondary,” etc. as used herein are intended for illustrative purposes only and do not limit the embodiments in any way. Further, the term “plurality” as used herein indicates any number greater than one, either disjunctively or conjunctively, as necessary, up to an infinite number.

It will be understood that the invention may be embodied in other specific forms without departing from the spirit or central characteristics thereof. The presenet examples and embodiments, therefore, are to be considered in all respects as illustrative and not restrictive, invention is not to be limited to the details given herein. Accordingly, while the embodiments have been illustrated and described, numerous modifications come to mind without significantly departing from the spirit of the invention and the scope of on is only limited by the scope of the accompanying Claims.

What is claimed is:

1. A surgical grasper comprising:
a shaft;
two jaws at a distal end of the shaft; and,
a strain gauge sensor that uses the Hall Effect to measure a force being applied by the jaws.

2. The surgical grasper of claim 1 wherein the strain gauge sensor is integrated with signal-conditioning electronics into a single chip or a single package sealed module.

3. The surgical grasper of claim 1, further comprising:
a microprocessor and a non-volatile memory chip for at least one of calibration parameter storage and forensic storage.

4. The surgical grasper of claim 1, further comprising:
a handle operably connected to the jaws, wherein the jaws can be actuated by the handle.

5. The surgical grasper of claim 4 wherein the strain gauge sensor is located on or inside the handle.

6. The surgical grasper of claim 1 wherein the strain gauge sensor is located on an inner surface of one or both of the jaws.

7. The surgical grasper of claim 1 for use in robotic surgery wherein the jaws can be actuated in response to a robot command.

8. The surgical grasper of claim 1 wherein the strain gauge sensor is located on or inside the shaft.

9. The surgical grasper of claim 7 wherein the strain gauge sensor is located at an actuator.

10. The surgical grasper of claim 7 wherein the strain gauge sensor is located on a wrist of a robot arm.

11. The surgical grasper of claim 7 wherein the measured force is fed back to the robot for use in adjusting the amount of force being applied by the jaws.

12. The surgical grasper of claim 1, further comprising:
a visual or audio signal corresponding to an amount of force being applied by the jaws.

13. A surgical grasper comprising:
a shaft;
two jaws at a distal end of the shaft; and,
a MEMS sensor.

14. The surgical grasper of claim 13 wherein the MEMS sensor is integrated with signal-conditioning electronics into a single chip or a single package sealed module.

15. The surgical grasper of claim 13, further comprising:
a microprocessor and a non-volatile memory chip for at least one of calibration parameter storage and forensic storage.

16. The surgical grasper of claim 13, further comprising:
a handle operably connected to the jaws, wherein the jaws can be actuated by the handle.

17. The surgical grasper of claim 16 wherein the MEMS sensor is located on or inside the handle.

18. The surgical grasper of claim 13, wherein the MEMS sensor is located on an inner surface of one or both of the jaws.

19. The surgical grasper of claim 13 for use in robotic surgery, wherein the jaws can be actuated in response to a robot command.

20. The surgical grasper of claim 13, wherein the MEMS sensor is located on or inside the shaft.

21. The surgical grasper of claim 19, wherein the MEMS sensor is located at an actuator.

22. The surgical grasper of claim 19, wherein the MEMS sensor is located on a wrist of a robot arm.

23. The surgical grasper of claim 19 wherein a measured value is fed back to the robot for use in adjusting the amount of force being applied by the jaws.

24. The surgical grasper of claim 13, further comprising:
a visual or audio signal corresponding to an amount of force being applied by the jaws.

25. A surgical feedback system comprising:
a surgical grasper capable of taking a force measurement, the grasper comprising: a shaft, two jaws at a distal end of the shaft, and a sensor; and,
a data concentrator coupled to the grasper via a wired or wireless interface using a first data transmission protocol with internal storage.

26. The surgical feedback system of claim 25 wherein the first data transmission protocol is selected from the group consisting of RS-232C, USB, Ethernet, Optical Fiber, Wireless USB, Wireless Ethernet, Firewire, Wi-Fi, 802.11B, 802.11g, Wi-Max, Wireless Telemetry and Bluetooth.

27. The surgical feedback system of claim 25 wherein the data concentrator is wireless and the force measurement is transmitted via the first data transmission protocol at least once every 100 milliseconds to the data concentrator.

28. The surgical feedback system of claim 25 wherein the data concentrator multiplexes a plurality of surgical graspers on a single data link to a monitoring station.

29. The surgical feedback system of claim 25, further comprising:
a visualizing display, a patient monitoring system, or a Hospital Information System coupled to the data concentrator via a wired or wireless interface and a second data transmission protocol for real-time and historical data transmission from the surgical grasper.

30. The surgical feedback system of claim 29 wherein the visualizing display, the patient monitoring system, or the Hospital Information System is selected from the group consisting of Analog, DVI, HDMI, Ethernet, Wireless Telemetry, Wi-Fi, Wi-Max, TCP/IP, Web Service, and HL7.

31. The surgical feedback system of claim 25 wherein the data concentrator stores a history of the force measurements for up to a given time of continuous operation for forensic purposes.

32. A method for measuring an amount of force being applied by the jaws of a grasper, the method comprising the steps of:
providing a grasper comprising a shaft and two jaws;
providing a strain gauge sensor; and,
providing for using the Hall Effect to measure an amount of force being applied to the strain gauge sensor.

33. The method of claim 32 wherein the strain gauge sensor is integrated with signal-conditioning electronics into a single chip or a single package sealed module.

34. The method of claim 32, further comprising the step of:
providing for calculating a pressure being applied by the jaws from the measured amount of force being applied to the strain gauge sensor.

35. The method of claim 34, further comprising the step of:
providing for visually displaying the calculated pressure.

36. The method of claim 32, further comprising the step of:
providing for sounding an audio alert corresponding to an amount of force being applied to the strain gauge sensor.

37. The method of claim 32, further comprising the step of:
providing a microprocessor and a non-volatile memory chip;
providing for storing calibration parameters in the memory chip at manufacturing time; and,
providing for storing the history of time-stamped transmitted data in the memory chip over a useful life of the grasper.

38. The method of claim 32, further comprising the step of:
providing a handle operably connected to the jaws, wherein the jaws can be actuated by the handle and the strain gauge sensor is provided on or inside the handle.

39. The method of claim 32 wherein the grasper is provided for use in robotic surgery and the jaws can be actuated responsive to a robot command.

40. The method of claim 39 wherein the strain gauge sensor is provided on or inside the shaft, on an inner surface of one or both of the jaws, at an actuator, or on a wrist of a robot arm.

41. The method of claim 39, further comprising the step of:
providing a feedback to the robot of the measured amount of force being applied to the strain gauge sensor for use in adjusting the amount of force being applied by the jaws.

42. A method for measuring an amount of force being applied by the jaws of a grasper, the method comprising the steps of:
providing a grasper comprising a shaft and two jaws; and, providing a MEMS sensor.

43. The method of claim 42 wherein the MEMS sensor is integrated with signal-conditioning electronics into a single chip or a single package sealed module.

44. The method of claim 42, further comprising the step of:
providing for calculating a pressure being applied by the jaws from the measured amount of force being applied to the MEMS sensor.

45. The method of claim 44, further comprising the step of:
providing for visually displaying the calculated pressure.

46. The method of claim 42, further comprising the step of:
providing for sounding an audio alert corresponding to an amount of force being applied to the MEMS sensor.

47. The method of claim 42, further comprising the step of:
providing a microprocessor and a non-volatile memory chip;
providing for storing calibration parameters in the memory chip at manufacturing time; and,
providing for storing the history of time-stamped transmitted data in the memory chip over a useful life of the grasper.

48. The method of claim 42, further comprising the step of:
providing a handle operably connected to the jaws, wherein the jaws can be actuated by the handle and the MEMS sensor is provided on or inside the handle.

49. The method of claim 42 wherein the grasper is provided for use in robotic surgery and the jaws can be actuated responsive to a robot command.

50. The method of claim 49 wherein the MEMS sensor is provided on or inside the shaft, on an inner surface of one or both of the jaws, at an actuator, or on a wrist of a robot arm.

51. The method of claim 49, further comprising the step of:
providing a feedback to the robot of the measured amount of force being applied to the MEMS sensor for use in adjusting the amount of force being applied by the jaws.

52. A method for obtaining surgical feedback, the method comprising the steps of:
providing a surgical grasper capable of taking a force measurement, the grasper comprising: a shaft, two jaws at a distal end of the shaft, and a sensor; and,
providing a data concentrator coupled to the grasper via a wired or wireless interface using a first data transmission protocol with internal storage.

53. The method of claim 52 wherein the first data transmission protocol is selected from the group consisting of RS-232C, USB, Ethernet, Optical Fiber, Wireless USB, Wireless Ethernet, Firewire, Wi-Fi, 802.11B, 802.11g, Wi-Max, Wireless Telemetry and Bluetooth.

54. The method of claim 52 wherein the data concentrator is wireless and the first data transmission protocol is capable of transmitting the force measurement to the data concentrator at least once every 100 milliseconds.

55. The method of claim 52 wherein the data concentrator is capable of multiplexing a plurality of surgical graspers on a single data link to a monitoring station.

56. The method of claim 52, further comprising the step of:
providing that the data concentrator is capable of being coupled to a visualizing display, a patient monitoring system, or a Hospital Information System via a wired or wireless interface and a second data transmission protocol for real-time and historical data transmission from the surgical grasper.

57. The method of claim 56 wherein the visualizing display, the patient monitoring system, or the Hospital Information System is selected from the group consisting of Analog, DVI, HDMI, Ethernet, Wireless Telemetry, Wi-Fi, Wi-Max, TCP/IP, Web Service, and HL7.

58. The method of claim 52 wherein the data concentrator stores a history of the force measurements for up to a given time of continuous operation for forensic purposes.
